(19) United States
(12) Patent Application Publication
(10) Pub. No.: US 2026/0219671 A1
(43) Pub. Date: Jul. 30, 2026

(54) METHOD FOR GRADUALLY TRAINING PRECISION MAGNETIC FIELD CONTROL SYSTEM FOR CONTROLLING THREE-DIMENSIONAL POSITION OF MICRO/NANO ROBOT

(71) Applicant: MIRACURE CO., LTD, Goyang-si (KR)

(72) Inventors: Hong Soo CHOI, Daegu (KR); Hak Joon LEE, Daegu (KR); Sarmad Ahmad ABBASI, Daegu (KR); Awais AHMED, Daegu (KR); Nader LATIFI GHARAMALEKI, Daegu (KR); Jin Young KIM, Daegu (KR)

(73) Assignee: MIRACURE CO., LTD, Goyang-si (KR)

(21) Appl. No.: 19/143,644

(22) PCT Filed: Dec. 21, 2023
(86) PCT No.: PCT/KR2023/021209

§ 371 Date: Jun. 26, 2025

(30) Foreign Application Priority Data
Dec. 26, 2022 (KR) 10-2022-0184525
Nov. 3, 2023 (KR) 10-2023-0150655

(51) Int. Cl. G05D 1/467; A61B 34/00; A61B 34/30; G05D 101/15; G05D 107/50; G06N 3/092; H02N 11/00

(52) U.S. Cl. CPC G05D 1/467; A61B 34/73; G06N 3/092; H02N 11/006; A61B 2034/302; G05D 2101/15; G05D 2107/50

(57) ABSTRACT

Disclosed is a method for training an artificial neural network capable of precision control of a magnetic robot. A method for gradually training an artificial neural network for controlling a magnetic robot, whereby the three-dimensional position of the magnetic robot is controlled, comprises: a primary training step in which a processor trains the artificial neural network to control the three-dimensional position of the magnetic robot in simulation environments; a secondary training step in which the processor additionally trains the artificial neural network to control the two-dimensional position of the magnetic robot in real-life environments after the primary training step; a tertiary training step in which the processor additionally trains the artificial neural network to control the three-dimensional position of the magnetic robot in real-life environments after the secondary training step; and a precision training step in which the processor additionally trains the artificial neural network to control the three-dimensional position of the magnetic robot in real-life environments having a preset restricted radius after the tertiary training step.

### TECHNICAL FIELD

The present disclosure relates to artificial intelligence training, and more specifically, to an artificial intelligence training method for precise movement control of a micro robot.

### BACKGROUND ART

The present application claims the benefit of Korean Patent Application No. 10-2022-0184525, filed in Korea on Dec. 26, 2022, and Korean Patent Application No. 10-2023-0150655, filed in Korea on Nov. 3, 2023, the entire contents of which are incorporated herein by reference.

The material described in this section merely provides background information on the embodiments described in the present disclosure and does not necessarily constitute prior art.

Parkinson's disease is considered one of the three major geriatric diseases along with dementia and stroke, and the number of patients is also increasing as the average age increases due to the aging society. According to the 2020 National Interest Disease Statistics of the Health Insurance Review and Assessment Service in Korea, the number of patients with Parkinson's disease by year is steadily increasing from 100,000 in 2015 to 125,000 in 2019 due to the aging society and westernized eating habits. Accordingly, the need for the development of new technologies for the treatment of Parkinson's disease is emerging.

Currently, in clinical practice, drug therapy that increases the concentration of dopamine and implantation surgery that uses electrical stimulation to treat the imbalance of the neural network are used to treat Parkinson's disease. However, drug therapy has problems such as instability of drug efficacy, hyperkinesia, and decreased drug efficacy, and surgical therapy has side effects such as infection, bleeding, stroke, and epilepsy.

Micro/nanorobots are expected to have the potential to dramatically overcome the side effects of existing drug treatments and the risks of surgical treatments as they are technologies that enable minimally invasive, targeted, and precise delivery of therapeutic agents. Cell therapy agents combined with magnetic nanoparticles may be transported to brain regions, and in particular, when the micro/nanorobots using magnetic nanoparticles are used, cell therapy agents or therapeutic drugs can be penetrated and controlled even into complex structures such as the brain. These medical micro/nanorobot-based neural network reconstruction platforms can improve existing treatment methods or may be used to develop completely new treatment techniques.

### DISCLOSURE

### Technical Problem

An object of the present disclosure is to provide a method for training an artificial neural network capable of precise control of a magnetic robot.

The present disclosure is not limited to the objects mentioned above, and other objects not mentioned will be clearly understood by those skilled in the art from the description below.

### Technical Solution

According to the present disclosure for solving the above-described problem, there is provided a method for gradually training an artificial neural network for controlling a three-dimensional position of a magnetic robot, the method including: a primary training step in which a processor trains the artificial neural network to control the three-dimensional position of the magnetic robot in a simulation environment; a secondary training step in which the processor additionally trains the artificial neural network to control a two-dimensional position of the magnetic robot in a real-life environment after the primary training step; a tertiary training step in which the processor additionally trains the artificial neural network to control the three-dimensional position of the magnetic robot in the real-life environment after the secondary training step; and a precision training step in which the processor additionally trains the artificial neural network to control the three-dimensional position of the magnetic robot in a real-life environment having a preset restricted radius after the tertiary training step.

According to one embodiment of the present disclosure, the artificial neural network may receive three coordinate values indicating a starting position of the magnetic robot and three coordinate values indicating a target position of the magnetic robot.

According to one embodiment of the present disclosure, the artificial neural network may output a current value or a driving motor control signal value that controls a magnetic field generated from a plurality of magnetic driving devices.

According to one embodiment of the present disclosure, the processor may perform training in which a reward or penalty is given according to whether the magnetic robot is at the target position or an error between an arrival position and the target position within a preset time at each training step.

According to one embodiment of the present disclosure, the processor may repeatedly perform training until an accumulated reward value at each training step reaches a preset target value.

A magnetic drive system according to the present disclosure include: a magnetic control unit having an artificial neural network that has completed training by the method for gradually training the artificial neural network for controlling the magnetic robot; and a plurality of magnetic driving devices.

According to one embodiment of the present disclosure, the magnetic driving device may be formed by an electromagnetic coil that generates an arbitrary magnetic field by current or a permanent magnet that generates an arbitrary magnetic field by a drive motor.

Other specific details of the present disclosure are included in the detailed description and drawings.

### Advantageous Effects

According to one aspect of the present disclosure, it is possible to precisely control magnetic micro/nanorobots. Accordingly, limitations of conventional drug/surgical treatments can be overcome, and minimally invasive, targeted precision treatment can be possible through micro/nanorobots. In particular, the present disclosure can be of great help to neural network reconstruction technology based on a magnetic micro/nanorobot system for overcoming Parkinson's disease and treating brain diseases.

According to another aspect of the present disclosure, due to the fast inference of the artificial neural network, a faster response speed can be achieved compared to the existing control algorithm.

According to another aspect of the present disclosure, since the nonlinearity of the artificial neural network effectively reflects the nonlinear section within the workspace, the present disclosure can have a wider workspace compared to the existing method.

According to another aspect of the present disclosure, the present disclosure can also be utilized in magnetic drive systems having different shapes and arrangements.

The effects of the present disclosure are not limited to the effects mentioned above, and other effects not mentioned will be clearly understood by those skilled in the art from the description below.

### DESCRIPTION OF DRAWINGS

FIG. 1 is a reference diagram for a magnetic drive system according to the present disclosure.

FIG. 2 is a reference diagram for a driving principle of a magnetic robot.

FIG. 3 is an exemplary configuration diagram of the magnetic drive system according to the present disclosure.

FIG. 4 is a reference diagram of an artificial neural network included in a magnetic control unit according to the present disclosure.

FIG. 5 is a schematic flowchart of a method for gradually training an artificial neural network for controlling a magnetic robot according to the present disclosure.

### MODE FOR DISCLOSURE

The advantages and features of the present disclosure disclosed in the present disclosure, and the methods for achieving them, will become apparent by referring to the embodiments described in detail below together with the accompanying drawings. However, the present disclosure is not limited to the embodiments disclosed below and may be implemented in various different forms, and the present embodiments are provided only to make the present disclosure complete and to fully inform those skilled in the art (hereinafter referred to as “ordinary skilled in the art”) of the scope of the present disclosure, and the scope of rights of the present disclosure is defined only by the scope of the claims.

The terminology used herein is for the purpose of describing embodiments only and is not intended to limit the scope of the present disclosure. In the present disclosure, the singular also includes the plural unless specifically stated otherwise. The terms “comprise” and/or “comprising” as used herein do not exclude the presence or addition of one or more other components in addition to the components mentioned.

Throughout the present disclosure, the same reference numerals refer to the same elements, and “and/or” includes each and every combination of one or more of the elements mentioned. Although “first”, “second”, or the like are used to describe various elements, it is to be understood that these elements are not limited by these terms. These terms are merely used to distinguish one element from another. Accordingly, it should be understood that a first element mentioned below may also be a second element within the technical scope of the present disclosure.

Unless otherwise defined, all terms (including technical and scientific terms) used in the present disclosure may be used with a meaning that can be commonly understood by those skilled in the art to which the present disclosure belongs. In addition, terms defined in commonly used dictionaries shall not be ideally or excessively interpreted unless explicitly specifically defined. Hereinafter, embodiments of the present disclosure will be described in detail with reference to the attached drawings.

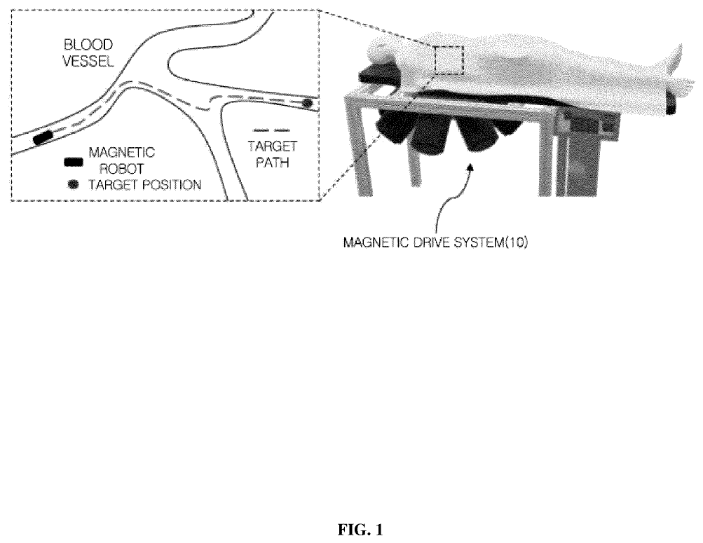

FIG. 1 is a reference diagram for a magnetic drive system according to the present disclosure.

Referring to FIG. 1, a magnetic drive system 10 according to the present disclosure and a patient lying thereon can be identified. The magnetic drive system 10 according to the present disclosure MAY be used in the medical field. Micro/nano robots MAY be used for the purpose of delivering therapeutic agents (drugs and cell therapy agents). For the accurate delivery of the therapeutic agent, precise movement control of the micro/nano robot should be possible from outside the patient. For this purpose, the micro/nano robot may be implemented as a “magnetic robot” that is influenced by a magnetic field.

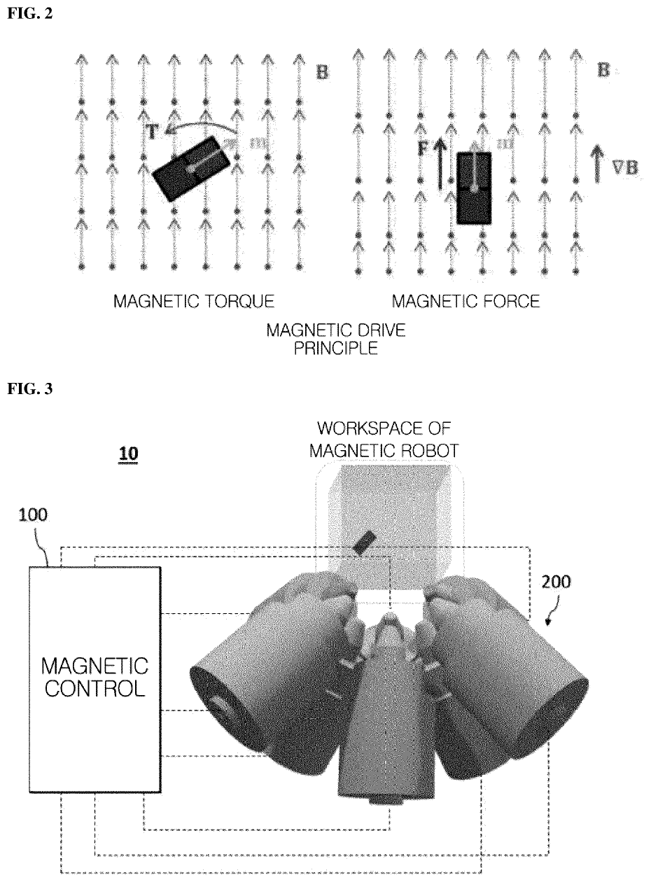

FIG. 2 is a reference diagram for the driving principle of the magnetic robot.

Referring to FIG. 2, a magnetic body may receive torque (rotation) or force (movement) by a magnetic field. Therefore, it is possible to implement a magnetic robot with a magnetic body and precisely adjust the magnetic robot by controlling the magnetic robot with a magnetic field. In particular, the magnetic field has excellent permeability to the human body and is biocompatible, so it is suitable for medical purposes.

FIG. 3 is an exemplary configuration diagram of the magnetic drive system according to the present disclosure.

Referring to FIG. 3, a magnetic drive system 10 according to the present disclosure may include a magnetic control unit 100 and a magnetic driving device 200.

The magnetic drive system 10 according to the present disclosure may include a plurality of magnetic driving devices 200. In the example illustrated in FIG. 3, eight magnetic driving devices 200 are illustrated, but the magnetic drive system 10 according to the present disclosure is not limited by the example. The magnetic driving device 200 may be classified into an electromagnetic coil type or a permanent magnet type. The electromagnetic coil type magnetic driving device 200 may generate an arbitrary magnetic field by current, and the permanent magnet type magnetic driving device 200 may generate an arbitrary magnetic field through physical movement such as up, down, left, right, or forward and backward by a drive motor. Therefore, the movement of the magnetic robot located in a workspace may be controlled by the magnetic fields respectively generated from the plurality of magnetic driving devices 200.

The magnetic control unit 100 may control each of the plurality of magnetic driving devices 200. When the magnetic driving device 200 is an electromagnetic coil type, the size of the magnetic field can be adjusted by controlling voltage/current, and when it is a permanent magnet type, the size of the magnetic field may be adjusted by controlling the operation of the drive motor. In order to precisely control the movement of the magnetic robot, the magnetic control unit 100 may have an artificial neural network. The artificial neural network is an artificial neural network trained by a training method according to the present disclosure, and may output voltage/current values or drive motor control signals so as to move the magnetic robot to a desired position.

In the present disclosure, for convenience of explanation, it will be assumed that all of the plurality of magnetic driving devices 200 are of the electromagnetic coil type. In addition, the description will be made on the premise that the artificial neural network outputs eight current values for controlling each of the magnetic driving devices.

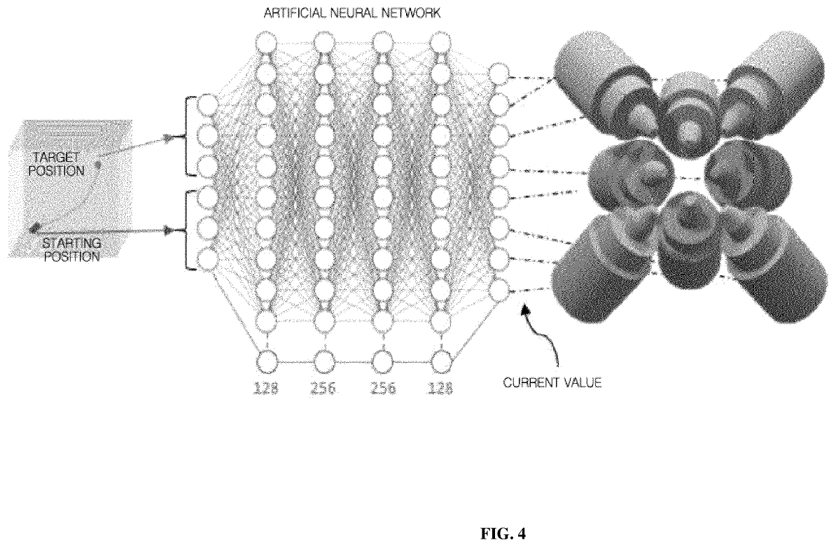

FIG. 4 is a reference diagram of the artificial neural network included in the magnetic control unit according to the present disclosure.

Referring to FIG. 4, it can be confirmed that the artificial neural network receives position information and outputs a value for controlling the magnetic driving device. The artificial neural network may receive information on the starting position and target position of the magnetic robot as input values. More specifically, the artificial neural network may receive three coordinate values (for example, x, y, and z) indicating the starting position of the magnetic robot and three coordinate values (for example, x, y, and z) indicating the target position of the magnetic robot. The output values may be eight current values, as described above. Meanwhile, the artificial neural network may include four hidden layers, and the first hidden layer may include 128 nodes, the second hidden layer may include 256 nodes, the third hidden layer may include 256 nodes, and the fourth hidden layer may include 128 nodes.

Below, a method for training the above artificial neural network will be described. As previously described, the above artificial neural network is an artificial neural network for controlling a magnetic robot to control the three-dimensional position of the magnetic robot. The characteristic of the training method according to the present disclosure is to gradually train the artificial neural network.

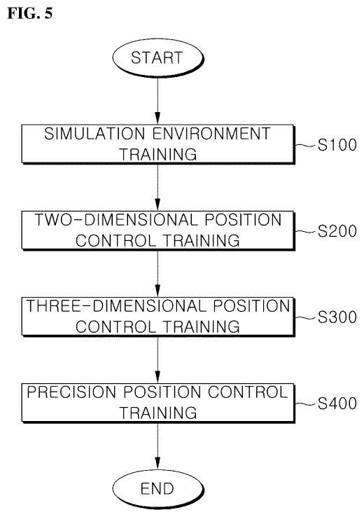

FIG. 5 is a schematic flowchart of a method for gradually training an artificial neural network for controlling a magnetic robot according to the present disclosure.

Referring to FIG. 5, the method for gradually training an artificial neural network for controlling a magnetic robot according to the present disclosure may include a simulation environment training step (S100), a two-dimensional position control training step (S200), a three-dimensional position control training step (S300), and a precise position control training step (S400).

First, in the simulation environment training step (S100), the processor may train the artificial neural network to control the three-dimensional position of the magnetic robot in a simulation environment. This step may be referred to as a “primary training step”.

After the primary training step, in the two-dimensional position control training step (S200), the processor may additionally train the artificial neural network to control the two-dimensional position of the magnetic robot in a real-life environment. This step may be referred to as a “secondary training step”. The secondary training step does not train a new artificial neural network, but additionally trains the artificial neural network that has completed the primary training step. In addition, since the secondary training step is performed in the real-life environment, unlike a simulation, it is possible to train the characteristics of an uneven magnetic field of an actual magnetic driving device 200. In the secondary training step, the training may be performed by inputting a coordinate value (for example, z axis) on any one axis of the three coordinate values (for example, x, y, and z) indicating the starting position of the magnetic robot and the three coordinate values (for example, x, y, and z) indicating the target position of the magnetic robot as “0”.

After the secondary training step, in the three-dimensional position control training step (S300), the processor may additionally train the artificial neural network to control the three-dimensional position of the magnetic robot in a real environment. This step may be referred to as a “tertiary training step”. The tertiary training step also does not train a new artificial neural network, but additionally trains the artificial neural network that completed the secondary training step.

After the tertiary training step, in the precision position control training step (S400), the processor may further train the artificial neural network to control the three-dimensional position of the magnetic robot in a real-life environment having a preset limit radius (for example, 200 micrometers). This step may be referred to as a “precision training step”.

The primary training step, secondary training step, tertiary training step, and precise training step may be performed by so-called “reinforcement learning” that trains an artificial intelligence model through rewards. The processor may perform training that gives a reward or penalty according to whether the magnetic robot is at the target position or the error between the arrival position and the target position within the preset time in each training step. In addition, the processor may repeat training until the accumulated reward value in each training step reaches a preset target value. When the accumulated reward value reaches the preset target value, the corresponding training step can be completed.

The magnetic control unit 100 may include a processor, an application-specific integrated circuit (ASIC), another chipset, a logic circuit, a register, a communication modem, a data processing device, or the like known in the technical field to which the present disclosure belongs in order to execute calculations and various control logics. In addition, when the above-described control logic is implemented as software, the processor may be implemented as a set of program modules. At this time, the program modules may be stored in the memory device and executed by the processor.

The above-mentioned computer program may include codes coded in a computer language, such as C/C++, C #, JAVA, Python, or machine language, that can be read by the processor (CPU) of the computer through the device interface of the computer, so that the computer reads the program and executes the methods implemented as a program. Such codes may include functional codes related to functions that define functions necessary for executing the methods, and may include control codes related to execution procedures necessary for the processor of the computer to execute the functions according to a predetermined procedure. In addition, such codes may further include memory reference-related codes regarding which location (address address) of the internal or external memory of the computer should be referenced for additional information or media necessary for the processor of the computer to execute the functions. In addition, when the processor of the computer needs to communicate with another computer or server located remotely in order to execute the functions, the code may further include communication-related code regarding how to communicate with another computer or server located remotely using the communication module of the computer, what information or media to send and receive during communication, or the like.

The storage medium means a medium that stores data semi-permanently and can be read by a device, rather than a medium that stores data for a short period of time, such as a register, cache, or memory. Specifically, examples of the storage medium include, but are not limited to, ROM, RAM, CD-ROM, magnetic tape, floppy disk, or optical data storage device. That is, the program can be stored in various storage media on various servers that the computer can access or in various storage media on the user's computer. In addition, the medium can be distributed to computer systems connected to a network, so that a computer-readable code can be stored in a distributed manner.

Although the embodiments of the present disclosure have been described above with reference to the attached drawings, it will be understood by those skilled in the art to which the present disclosure pertains that the present disclosure may be implemented in other specific forms without changing the technical idea or essential characteristics thereof. Therefore, it should be understood that the embodiments described above are illustrative in all respects and not restrictive.

### DETAILED DESCRIPTION OF MAIN ELEMENTS

10: magnetic drive system
100: magnetic control unit
200: magnetic driving device

1. A method for gradually training an artificial neural network for controlling a three-dimensional position of a magnetic robot, the method comprising:
a primary training step in which a processor trains the artificial neural network to control the three-dimensional position of the magnetic robot in a simulation environment;
a secondary training step in which the processor additionally trains the artificial neural network to control a two-dimensional position of the magnetic robot in a real-life environment after the primary training step;
a tertiary training step in which the processor additionally trains the artificial neural network to control the three-dimensional position of the magnetic robot in the real-life environment after the secondary training step; and
a precision training step in which the processor additionally trains the artificial neural network to control the three-dimensional position of the magnetic robot in a real-life environment having a preset restricted radius after the tertiary training step.

2. The method of claim 1, wherein the artificial neural network receives three coordinate values indicating a starting position of the magnetic robot and three coordinate values indicating a target position of the magnetic robot.

3. The method of claim 1, wherein the artificial neural network outputs a current value or a driving motor control signal value that controls a magnetic field generated from a plurality of magnetic driving devices.

4. The method of claim 1, wherein the processor performs training in which a reward or penalty is given according to whether the magnetic robot is at the target position or an error between an arrival position and the target position within a preset time at each training step.

5. The method of claim 4, wherein the processor repeatedly performs training until an accumulated reward value at each training step reaches a preset target value.

6. A magnetic drive system comprising:
a magnetic control unit having an artificial neural network that has completed training by the method for gradually training according to claim 1; and
a plurality of magnetic driving devices.

7. The magnetic drive system of claim 6, wherein the magnetic driving device is formed by an electromagnetic coil that generates an arbitrary magnetic field by current or a permanent magnet that generates an arbitrary magnetic field by a drive motor.

8. A magnetic drive system comprising:
a magnetic control unit having an artificial neural network that has completed training by the method for gradually training according to claim 2; and
a plurality of magnetic driving devices.

9. A magnetic drive system comprising:
a magnetic control unit having an artificial neural network that has completed training by the method for gradually training according to claim 3; and
a plurality of magnetic driving devices.

10. A magnetic drive system comprising:
a magnetic control unit having an artificial neural network that has completed training by the method for gradually training according to claim 4; and
a plurality of magnetic driving devices.

11. A magnetic drive system comprising:
a magnetic control unit having an artificial neural network that has completed training by the method for gradually training according to claim 5; and
a plurality of magnetic driving devices.

12. The magnetic drive system of claim 8, wherein the magnetic driving device is formed by an electromagnetic coil that generates an arbitrary magnetic field by current or a permanent magnet that generates an arbitrary magnetic field by a drive motor.

13. The magnetic drive system of claim 9, wherein the magnetic driving device is formed by an electromagnetic coil that generates an arbitrary magnetic field by current or a permanent magnet that generates an arbitrary magnetic field by a drive motor.

14. The magnetic drive system of claim 10, wherein the magnetic driving device is formed by an electromagnetic coil that generates an arbitrary magnetic field by current or a permanent magnet that generates an arbitrary magnetic field by a drive motor.

15. The magnetic drive system of claim 11, wherein the magnetic driving device is formed by an electromagnetic coil that generates an arbitrary magnetic field by current or a permanent magnet that generates an arbitrary magnetic field by a drive motor.
